(19) United States
(12) Patent Application Publication
(10) Pub. No.: US 2015/0272674 A1
(43) Pub. Date: Oct. 1, 2015

(54) DUAL WAVELENGTH LASER LITHOTRIPSY

(71) Applicant: AMS RESEARCH CORPORATION, Minnetonka, MN (US)

(72) Inventors: Rongwei Jason Xuan, Fremont, CA (US); Thomas Charles Hasenberg, Campbell, CA (US); Jian James Zhang, S. Setauket, NY (US)

(73) Assignee: AMS RESEARCH CORPORATION, Minnetonka, MN (US)

(21) Appl. No.: 14/437,451

(22) PCT Filed: Nov. 6, 2013
(86) PCT No.: PCT/US2013/068653

§ 371 Date: Apr. 21, 2015

Related U.S. Application Data
(60) Provisional application No. 61/723,822, filed on Nov. 8, 2012.

(51) Int. Cl. A61B 18/20; A61B 18/08

(52) U.S. Cl. CPC A61B 18/20; A61B 18/082; A61B 2018/206

(57) ABSTRACT

A laser lithotripsy method for fragmenting a kidney or bladder stone in a patient is provided. The method includes delivering a first laser energy having a first wavelength to the stone. The stone is heated in response to the delivery of the first laser energy to the stone. The method also includes delivering a second laser energy to the stone having a second wavelength that has a higher absorption by the stone or the fluid surrounding the stone than the first wavelength. The stone is fragmented in response to the delivery of the second laser energy to the stone.

### BACKGROUND

The present invention relates generally to laser lithotripsy, and more particularly to a method of laser lithotripsy using dual wavelength laser energy having a wavelength with less absorption by the stone to heat the stone first and then a stronger absorption wavelength by the stones or the fluid near the stones to break the stones.

The treatment of kidney or bladder calculi or stones or other stones or calculi within the human body, lithotripsy, is currently achieved through either ESWL (extra-corporal sound wave lithotripsy), or surgery, or use of a laser (laser lithotripsy). In recent years, cases of stone disease treatment by laser lithotripsy have surpassed ESWL. For laser lithotripsy, the Holmium:YAG (Ho:YAG) laser with a wavelength of around 2100 nm has become the standard choice for laser lithotripsy of all stone types. Laser lithotripsy fragmentation processes have been discussed by Kin Foong Chan, et al., in Journal of Endourology Vol. 15, number 3, pp 257-273 (2001) and many others.

Detailed studies have shown that the fragmentation process in Ho:YAG laser lithotripsy was predominantly photothermal, secondary to a long pulse duration that significantly reduced the strength of acoustic emission. The vapor bubble produced an open channel that facilitated laser delivery to the calculus surface (Moses effect). Light absorption within the calculus caused a rapid temperature rise above the threshold for chemical breakdown, resulting in calculus decomposition and fragmentation. With a Q-switched laser, the laser pulse duration is normally in the nanosecond (ns) range. This laser pulse can generate plasma with temperatures over 6000K (M. E. Mayo, PhD Thesis, pp 120-126, Cranfield University, UK 2009). This Q-switched laser pulse can lead to thermo-mechanical (mechanical confined) effect that breaks up the calculus. This super heated area will generate vaporized bubbles that can create shockwaves during the collapse of the bubble. The shockwave fragments the calculus in its vicinity.

### SUMMARY

Some embodiments of the invention are directed to a laser lithotripsy method for fragmenting a kidney or bladder stone in a patient. In the method, a first laser energy having a first wavelength is delivered to the stone. The stone is heated in response to the delivery of the first laser energy to the stone. A second laser energy is then delivered to the stone having a second wavelength that has a higher absorption by the stone or the fluid surrounding the stone than the first wave length. The stone is fragmented in response to the delivery of the second laser energy to the stone.

Some embodiments are directed to a method of fragmenting a calculus in a patient. In the method, a first laser energy having a first wavelength is delivered to the calculus. The calculus is heated in response to the delivery of the first laser energy to the calculus. A second laser energy is then delivered to the calculus having a second wavelength that has a higher absorption by the calculus or the fluid surrounding the calculus than the first wave length. A shockwave is generated in response to the delivery of the second laser energy to the calculus. The calculus is fragmented in response to the shockwave.

Some embodiments are directed to a method of fragmenting a calculus at a treatment site in a patient. In the method, a laser system is provided that comprises a first laser source, a second laser source, a controller and a laser fiber having a longitudinal axis. The first laser source generates a first laser energy having a first wavelength. The second laser source generates a second laser energy having a second wavelength with a higher absorption by the calculus or the fluid surrounding the calculus than the first wavelength. In the method, the first laser energy is delivered to the calculus. The calculus is heated in response to the delivery of the first laser energy to the calculus. The second laser energy is then delivered to the calculus. The calculus is fragmented in response to the delivery of the second laser energy to the calculus.

In some embodiments, the first laser energy has an energy level in the range of 0.01-10 J or 0.001-10 J. In some embodiments, the second laser energy has an energy level in the range of 0.01-10 J or 0.001-10 J.

### BRIEF DESCRIPTION OF THE DRAWINGS

FIG. 1 is a schematic diagram of an exemplary surgical laser system in accordance with embodiments of the invention.

FIG. 2 is a flowchart illustrating a laser lithotripsy method in accordance with embodiments of the invention.

FIGS. 3-5 are simplified illustrations of various steps of the method.

### DETAILED DESCRIPTION OF ILLUSTRATIVE EMBODIMENTS

Embodiments of the invention are described more fully hereinafter with reference to the accompanying drawings. Elements that are identified using the same or similar reference characters refer to the same or similar elements. The various embodiments of the invention may, however, be embodied in many different forms and should not be construed as limited to the embodiments set forth herein. Rather, these embodiments are provided so that this disclosure will be thorough and complete, and will fully convey the scope of the invention to those skilled in the art.

Specific details are given in the following description to provide a thorough understanding of the embodiments. However, it is understood by those of ordinary skill in the art that the embodiments may be practiced without these specific details. For example, circuits, systems, networks, processes, frames, supports, connectors, motors, processors, and other components may not be shown, or shown in block diagram form in order to not obscure the embodiments in unnecessary detail.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

It will be understood that when an element is referred to as being “connected” or “coupled” to another element, it can be directly connected or coupled to the other element or intervening elements may be present. In contrast, if an element is referred to as being “directly connected” or “directly coupled” to another element, there are no intervening elements present.

It will be understood that, although the terms first, second, etc. may be used herein to describe various elements, these elements should not be limited by these terms. These terms are only used to distinguish one element from another. Thus, a first element could be termed a second element without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As will further be appreciated by one of skill in the art, the present invention may be embodied as methods, systems, and/or computer program products. Accordingly, the present invention may take the form of an entirely hardware embodiment, an entirely software embodiment or an embodiment combining software and hardware aspects. Furthermore, the present invention may take the form of a computer program product on a computer-usable storage medium having computer-usable program code embodied in the medium. Any suitable computer readable medium may be utilized including hard disks, CD-ROMs, optical storage devices, or magnetic storage devices. Such computer readable media and memory for computer programs and software do not include transitory waves or signals.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the invention may also be described using flowchart illustrations and block diagrams. Although a flowchart may describe the operations as a sequential process, many of the operations can be performed in parallel or concurrently. In addition, the order of the operations may be re-arranged. A process is terminated when its operations are completed, but could have additional steps not included in a figure or described herein.

It is understood that one or more of the blocks (of the flowcharts and block diagrams) may be implemented by computer program instructions. These program instructions may be provided to a processor circuit, such as a microprocessor, microcontroller or other processor, which executes the instructions to implement the functions specified in the block or blocks through a series of operational steps to be performed by the processor(s) and corresponding hardware components.

Embodiments of the present invention relate to a method of performing laser lithotripsy that generally involves a two-stage process. The first stage of the method is a heating stage, in which a targeted stone is heated using laser energy of a first wavelength, which has a low absorption by the stone. In the second stage, laser energy having a second wavelength, which has stronger absorption by the stone than the first wavelength, is directed at the stone to break the stone into fragments. Embodiments of the present invention can be used to treat all types of stones or calculi within the human body including, but not limited to, kidney stones, bladder stones, prostate stones and gall stones, and can also be used in the treatment of urolithiasis.

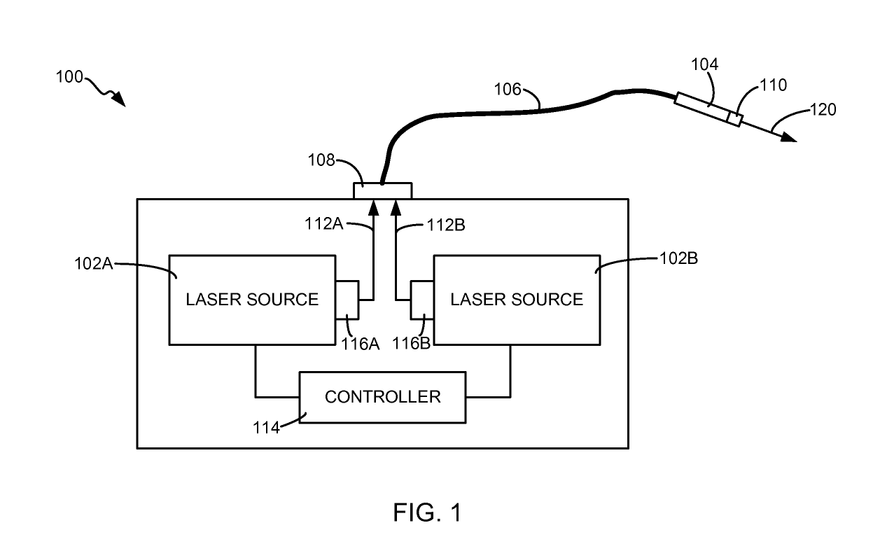

FIG. 1 is a schematic diagram of an exemplary surgical laser system 100 in accordance with embodiments of the invention. In one embodiment, the system 100 includes laser sources 102A and 102B and a laser fiber 104. Each of the laser sources 102 generates electromagnetic radiation or laser energy in the form of a laser beam in accordance with conventional techniques. The laser fiber 104 includes a waveguide 106 that is coupled to the laser energy generated by the laser source 102 through a suitable optical coupling 108. The laser fiber 104 includes a probe tip 110 where the laser energy 112 is discharged to a desired laser treatment site. Embodiments of the probe tip 110 are configured to discharge the laser energy laterally relative to a longitudinal axis of the laser fiber 104, such as with an acute angle relative to a longitudinal axis of the laser fiber 104 (side-firing), and/or substantially along the longitudinal axis of laser fiber 104 (end-firing), as shown in FIG. 1. The laser fiber 104 may be supported by an endoscope or cystoscope during laser treatments in accordance with conventional techniques.

In one embodiment, the system 100 includes a controller 114 that includes one or more processors that are configured to execute program instructions stored in memory of the system 100 and perform various functions in accordance with embodiments described herein in response to the execution of the program instructions. These functions include, for example, the control of the laser sources 102 and the generation and delivery of laser energy 112 through the laser fiber 104, and other functions.

The laser sources 102A and 102B are conventional laser sources, such as laser resonators, that are configured to respectively generate laser energy 112A and 112B, as illustrated in FIG. 1. Shutter mechanisms 116A and 116B respectively control the discharge of the laser energy 112A and 112B. The shutter mechanisms 116 may be controlled by the controller 114 in response to an input from the physician, such as through a foot pedal or other input device in accordance with conventional surgical laser systems.

In one embodiment, the laser energy 112A generated by the laser source 102A has a wavelength with less stone absorption than the laser energy 112B generated by the laser source 102B. In some embodiments, the laser energy 112A has a shorter wavelength than the laser energy 112B. In other embodiments, the laser energy 112A has a longer wavelength than the laser energy 112B. In some embodiments, the laser energy 112A has a lower power than the laser energy 112B.

In one embodiment, the laser energy 112A is configured to be absorbed by kidney or bladder stones to heat the kidney or bladder stones. In one embodiment, the laser energy 112A is configured to penetrate kidney or bladder stones to a greater depth than the laser energy 112B. In some embodiments, the laser energy 112A has a wavelength in the range of 550-11000 nm. In one preferred embodiment, the wavelength of the laser energy 112A is approximately 1064 nm. Embodiments also include other wavelengths for the laser energy 112A. In some embodiments, the laser energy 112A has an energy level in the range of 0.01-10 J or 0.001-10 J. Suitable laser sources configured to generate the laser energy 112A include, for example, Nd:YAG, Nd:YLF, Nd:YVO₄, Yb:YAG, etc.

The laser energy 112B generated by the laser source 102B serves the purpose of fragmenting the kidney or bladder stone after the stone has been heated using the laser energy 112A. The laser energy 112B generally has a shorter or longer wavelength, higher absorption by the stone(s) or the fluid surrounding the stone(s) and a higher peak power than the laser energy 112A. In one embodiment, the laser energy 112B has a wavelength in the range of 200-550 nm or 1300 nm to 11000 nm. Embodiments also include other wavelengths for the laser energy 112B. In one preferred embodiment, the laser energy 112B has a wavelength of approximately 532 nm or 2.1 um and 2.01 um. In some embodiments, the laser energy 112B has an energy level in the range of 0.01-10 J or 0.001-10 J.

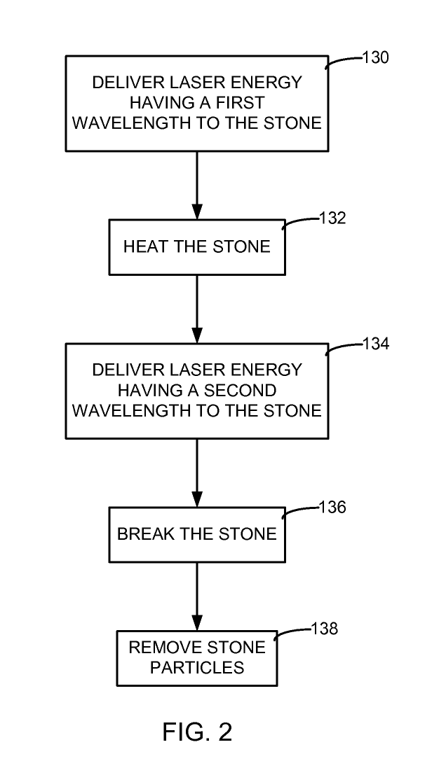

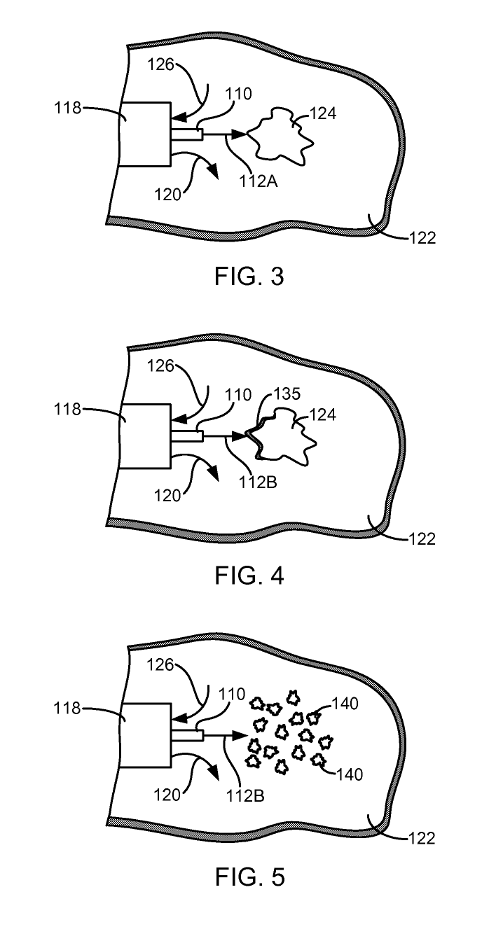

FIG. 2 is a flowchart of a laser lithotripsy method using the surgical laser system 100 in accordance with embodiments of the invention. Reference will be made to FIGS. 3-5, which are simplified illustrations of various steps of the method. In FIGS. 3-5, the laser fiber 104 and probe tip 110 are illustrated as being supported in an endoscope or a cystoscope 118, through which a flow of irrigant 120 may be introduced into the cavity 122, in which the targeted kidney or bladder stone 124 is located. Additionally, a flow of fluid and debris 126 may also be removed from the patient through the endoscope or a cystoscope 118 using conventional techniques.

At 130 of the method, laser energy 112A generated by the laser source 102A having a first wavelength is delivered to the stone 124. The laser energy 112A is generated by the laser source 102A and is in accordance with one or more of the embodiments described above. At 132 of the method, laser energy 112A is absorbed by the stone 124 thereby heating the stone 124 in response to exposure to the laser energy 112A.

At 134 of the method, laser energy 112B generated by the laser source 102B having a second wavelength that has a stronger absorption by the stone 124 than the first wavelength is delivered to the stone 124 through the laser fiber 104, as illustrated in FIG. 4. In one embodiment, the laser energy 112B is generated by the laser source 102B and is in accordance with one or more of the embodiments described above.

In one embodiment, the discharge of the laser energy 112A is terminated prior to the discharge of the laser energy 112B through, for example, control of the shutter mechanisms 116A and 116B by the controller 114. In accordance with another embodiment, the discharge of the laser energy 112B begins a short time prior to the termination of the discharge of the laser energy 112A. That is, in one embodiment, at the onset of step 134, the stone 124 is exposed to both laser energy 112A and laser energy 112B for a short period of time, such as 10⁻⁹-10⁻³ seconds.

In one embodiment, the surface of the stone 124 that is exposed to the laser energy 112B is heated relative to the remainder of the stone 124 because of the high absorption characteristics of the laser energy 112B wavelength by the stone 124 or fluid surrounding the stone 124. In one embodiment, a high temperature plasma formation 135 is formed on or adjacent to the exposed surface of the stone 124 in response to exposure of the stone 124, or the fluid surrounding the stone 124, to the laser energy 112B, as illustrated in FIG. 4.

At 136 of the method, the stone 124 is broken or fragmented in response to exposure to the laser energy 112B in step 134, as illustrated in FIG. 5. This breaking or fragmenting of the stone 124 occurs as a result of a mechanical shockwave that is generated in the high temperature plasma layer 135 due to pre-heating the stone 124 with laser energy 112A and then exposing the stone 124 to laser energy 112B. At 138 of the method, the stone fragments 140 are removed from the patient, such as through the recovered fluid and debris represented by arrow 126.

Although the present invention has been described with reference to preferred embodiments, workers skilled in the art will recognize that changes may be made in form and detail without departing from the spirit and scope of the invention.

1. A laser lithotripsy method for fragmenting a kidney or bladder stone in a patient comprising:
delivering a first laser energy having a first wavelength to the stone;
heating the stone in response to delivering the first laser energy to the stone;
delivering a second laser energy to the stone having a second wavelength that has a higher absorption by the stone or the fluid surrounding the stone than the first wavelength; and
fragmenting the stone in response to delivering second laser energy to the stone.

2. The method of claim 1, wherein the first wavelength is in the range of approximately 550-11,000 nm.

3. The method of claim 1, wherein the second wavelength is in the range of approximately 200-550 nm or 1300 nm to 11,000 nm.

4. The method of claim 1, wherein the first laser energy has an energy level of approximately 0.001-10 J.

5. The method of claim 1, wherein the second laser energy has an energy level of approximately 0.001-10 J.

6. The method of claim 1, wherein delivering second laser energy overlaps delivering first laser energy for a limited period of time.

7. The method of claim 1, wherein delivering second laser energy does not overlap delivering first laser energy.

8. A method of fragmenting a calculus in a patient comprising:
delivering a first laser energy having a first wavelength to the calculus;
heating the calculus in response to delivering the first laser energy to the calculus;
delivering a second laser energy to the calculus having a second wavelength that has a higher absorption by the calculus or the fluid surrounding the calculus than the first wavelength;
generating a shockwave in response to delivering the second laser energy to the calculus; and
fragmenting the calculus in response to the shockwave.

9. The method of claim 8, wherein the first wavelength is in the range of approximately 550-11000 nm.

10. The method of claim 8, wherein the second wavelength is in the range of approximately 200-550 nm or 1300 nm to 11000 nm.

11-18. (canceled)

19. A surgical laser apparatus for fragmenting a human calculus comprising:
a first laser source configured to generate first laser energy having a first wavelength;
a second laser source configured to generate second laser energy having a second wavelength that is different from the first wavelength;
a laser fiber comprising a waveguide and a probe tip at a distal end of the waveguide, the waveguide configured to deliver the first laser energy and the second laser energy to the probe tip, which discharges the first laser energy and the second laser energy;
wherein the first laser energy is configured to heat the calculus, and the second laser energy is configured to fragment the calculus.

20. The surgical laser apparatus according to claim 19, wherein the second wavelength is more absorbable by the calculus than the first wavelength.

21. The surgical laser apparatus according claim 19, wherein the second wavelength is shorter than the first wavelength.

22. The surgical laser apparatus according to claim 19, wherein the first wavelength is in the range of approximately 550-11,000 nm.

23. The surgical laser apparatus according to claim 19, wherein the second wavelength is in the range of approximately 200-550 nm.

24. The surgical laser apparatus according to claim 20, wherein the second wavelength is longer than the first wavelength.

25. The surgical laser apparatus of claim 19, wherein the first laser energy has a lower energy level than the second laser energy.

26. The surgical laser apparatus according to claim 19, wherein:
the first laser source includes a shutter mechanism that controls the discharge of the first laser energy to the laser fiber;
the second laser source includes a shutter mechanism that controls the discharge of the second laser energy to the laser fiber; and
the apparatus includes a controller configured to control the shutter mechanisms of the first and second laser sources.

27. The surgical laser apparatus of claim 26, wherein, for a first period of time, the controller controls the shutter mechanisms of the first and second laser sources to deliver the first laser energy to the laser fiber and block the delivery of the second laser energy to the laser fiber.

28. The surgical laser apparatus of claim 26, wherein, for a second period of time, the controller controls the shutter mechanisms of the first and second laser sources to deliver the second laser energy to the laser fiber and block the delivery of the first laser energy to the laser fiber.
